(19) United States
(12) Patent Application Publication
(10) Pub. No.: US 2018/0125694 A1
(43) Pub. Date: May 10, 2018

(54) Mouse Hole Finger Band

(71) Applicant: Kecia Elette Flournoy, Bellwood, IL (US)

(72) Inventor: Kecia Elette Flournoy, Bellwood, IL (US)

(21) Appl. No.: 15/346,704

(22) Filed: Nov. 8, 2016

(51) Int. Cl. A61F 5/058

(52) U.S. Cl. CPC A61F 5/05875

(57) ABSTRACT

A stretchable band placed over the 4^{th} and 5^{th} fingers resting between the base of the fingers and/or over or just at the knuckles of these fingers, that is used to support and/or stabilize these fingers while using a computer mouse, and/or other electronic devices.

### CROSS-REFERENCE TO RELATED APPLICATIONS

A provisional application for patent was submitted with the filing date Nov. 6, 2016 with the application No. 62/285,727 by inventor Kecia Elette Flournoy. The contents of which are entirely incorporated herein by reference.

### STATEMENT REGARDING FEDERALLY SPONSORED RESEARCH OR DEVELOPMENT (IF APPLICABLE)

“Not Applicable”

### REFERENCE TO SEQUENCE LISTING, A TABLE, OR A COMPUTER PROGRAM LISTING COMPACT DISC APPENDIX (IF APPLICABLE)

“Not Applicable”

### BACKGROUND OF THE INVENTION

A band is typically fashioned in the form of a loop and/or ring and is often constructed from a flat and/or thin strip of a hard opaque, shiny material, such as metal or precious metals with the ends fused together to form a circle. A band can also be made from a more flexible, stretchable type material such as elastic polymers or other viscoelastic type materials with the ends attached to make a circular form. Bands are frequently used in the aid of some medical conditions by providing pain relief and/or support of limbs, joints and/or bones. A band provides comfort and support by surrounding and compressing what is to be supported. Bands can be used to keep something together, bear weight, hold something up or used as a decoration and/or declaration such as with a wedding band. This invention addresses the need to stabilize the 4^{th} and 5^{th} fingers, while using a computer mouse or other electronic and/or touch screen devices to provide comfort and lessen the stiffen feeling associated with repeated use of these devices and the redundancy of movement involved.

### BRIEF SUMMARY OF THE INVENTION

The present invention generally relates to some of the issue we face from daily repeated use of electronic devices included, but not limited to PC and/or desktop which require a computer mouse, video games, cellphones and tablets and other touchscreen devices. Because of the specific redundant and/or repetitive movement involved while using these devices the 4^{th} and 5^{th} fingers are left in an unnatural position which can cause stiffness in these fingers. This invention will help to alleviate, lessen and in some cases, prevent this discomfort by placing these fingers in a more relaxed position.

### BRIEF DESCRIPTION OF THE VIEWS OF THE DRAWINGS

FIG. 1 illustrates a palm side down view of the finger band in position;

FIG. 2 illustrates a palm side up view of the finger band in position;

FIG. 3 illustrates an enlarged cross-sectional view of the finger band.

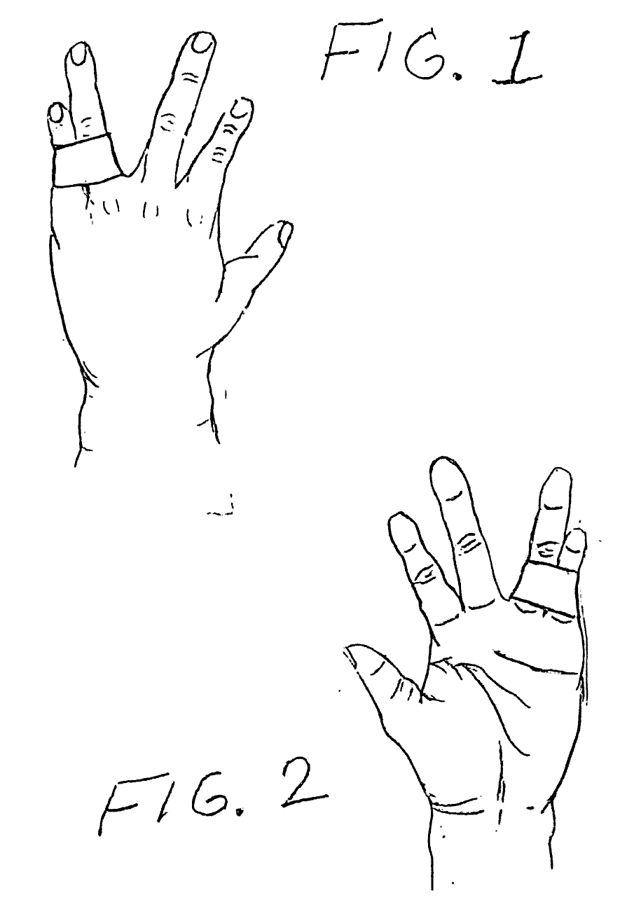

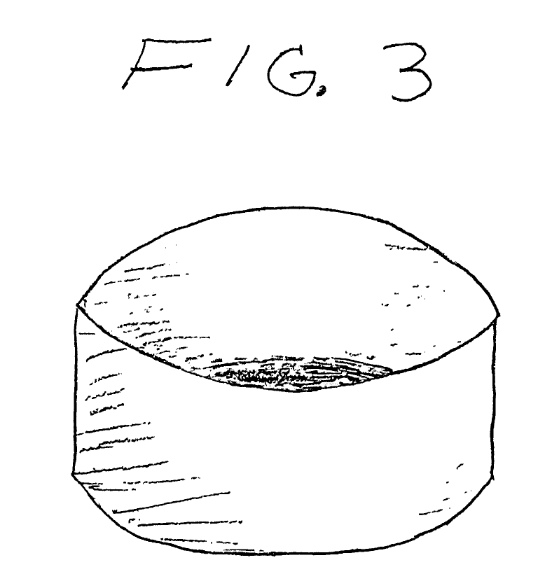

### DETAILED DESCRIPTION OF THE INVENTION

The invention Mouse Hole Finger Band is to be referred to here on throughout the remainder of this document and/or application simply as the finger band for the purposes of clarity and simplicity only. It is not to denounce or alter the name of the invention in anyway.

The finger band is used to support and/or stabilize the 4^{th} and 5^{th} fingers to help alleviate, lessen and/or and in some cases, prevent the stiffening feeling which commonly occurs with the repetitive and/or redundant movements involved in the use of a computer mouse, electronic and/or touch screen devices. The finger band is a made of a stretchable, flexible, breathable type material such as elastic polymers or other viscoelastic type materials fashioned in a hollow circular form such as the form of a ring and is often constructed from a flat and/or thin strip with the ends attached to form a circle.

Because it is flexible and stretchable and can be constructed in a variety of sizes, the exact position will depend on the size, length and width of the individual's fingers and the size of the band in use. To position the finger, band the user holds the 4^{th} and 5^{th} fingers together and slips the band down to the base of the fingers. When positioned and sized correctly the finger band should not interfere with the flexibility of the knuckles.

The finger band is not designed to prevent, reverse, alleviate or slow down any medical condition. To care for the finger band hand wash it with soap and warm water then place on a cloth to dry.

I claim this invention:

1. A finger band comprising:
A strip of stretchable, flexible, lightweight material in the form of a hollow circular shape that is positioned on the 4^{th} and 5^{th} fingers, just at or slightly beneath the knuckles down to the base of the fingers.

2. Used to support and/or stabilize the 4^{th} and 5^{th} fingers while using a computer mouse, electronic and/or touch screen devices.
